(19) United States
(12) Patent Application Publication
(10) Pub. No.: US 2013/0052137 A1
(43) Pub. Date: Feb. 28, 2013

(54) PARA AMINO BENZOIC ACID BASED NANOTUBES

(71) Applicants: Jhillu S. Yadav, Hyderabad (IN); Madugula Lavanya, Hyderabad (IN); Pragna P. Das, Hyderabad (IN); Anita Krishnan, Hyderabad (IN); Bulusu Jagannadh, Hyderabad (IN); Manika Pal-Bhadra, Hyderabad (IN); Utpal Bhadra, Hyderabad (IN)

(72) Inventors: Jhillu S. Yadav, Hyderabad (IN); Madugula Lavanya, Hyderabad (IN); Pragna P. Das, Hyderabad (IN); Anita Krishnan, Hyderabad (IN); Bulusu Jagannadh, Hyderabad (IN); Manika Pal-Bhadra, Hyderabad (IN); Utpal Bhadra, Hyderabad (IN)

(73) Assignees: COUNCIL OF SCIENTIFIC AND INDUSTRIAL RESEARCH, New Delhi (IN); INDIAN INSTITUTE OF CHEMICAL TECHNOLOGY, Hyderabad, Andhra Pradesh (IN)

(21) Appl. No.: 13/381,495

(22) PCT Filed: Jun. 29, 2010
(86) PCT No.: PCT/IN2010/000443

§ 371 Date: Sep. 18, 2012

(30) Foreign Application Priority Data
Jul. 3, 2009 (IN) 1584/CHE/2009

(51) Int. Cl. C07D 213/75; C07D 405/10; A61K 49/00; B82Y 5/00

(52) U.S. Cl. 424/9.6; 546/309; 546/282.7; 977/762; 977/904

(57) ABSTRACT

The invention relates to nanotubes and processes for making them and intermediates of a drug moiety or its derivative/s as a structural moiety with side chain/s capable of promoting self aggregation. The side chain/s are mono or a multiple of alkyl chain/s that are substituted or unsubstituted capable of creating self aggregating assemblies. A p-amino benzoic acid (PABA) moiety or derivative as a structural moiety of 4-alkylamido-N-pyridin-2yl-benzamide with a lauric or stearic side chain. 4-alkylamido-N-pyridin-2yl-benzamide and 4-Amino-N-pyridine-2 yl-benzamide, N-(Pyridine-2-yl)-4-dodecanamido-benzamide and N-(Pyridin-2-yl)-4-stearamidobenzamide are intermediates useful for in vivo Conjugation of an active ingredient, illustrated by Rhodamine B. With Rhodamine B as an active ingredient, nano-tubes prepared with lauric side chain, which show red fluorescence and is useful for in vivo tracking.

This is a National Phase application filed under 35 U.S.C. 371 as a national stage of PCT/IN2010/000443, filed Jun. 29, 2010, an application claiming the benefit from Indian Application No. 1584/CHE/2009, filed Jul. 3, 2009, the content of each of which is hereby incorporated by reference in its entirety.

### TECHNICAL FIELD

The invention relates to preparation of nanotubes from a drug moiety as structural component as illustrated by p-aminobenzoic acid moiety.

Particularly, the invention relates to preparation of nanotubes for oral delivery from p-amino benzoic acid, particularly as a drug delivery vehicle, molecular diagnostic and therapeutic use against all diseases.

The invention also relates to applications in agriculture and veterinary medicine.

### BACKGROUND OF INVENTION

Nanostructures, such as nanotubes, have been a subject of great interest for a variety of reasons that includes their use as drug delivery devices.

Major focus in the development of nanostructure for biomaterial delivery relies on three important factors, chemical modification, biocompatibility and minimal damage of the harbouring environment. Several novel materials have recently been used^{1,2} that show a greater promise in nano industries including quantum dots ^{3,4}, inorganic nanotubes (carbon, gold particles) ^{5,6}, organic polymers ⁷, bi- or multilayer liposome ^{6,9}, magnetofluorescent nanoparticles ^{10,11} etc. They show great promise, but each material has its own limitations in biological systems. The biggest obstacles in selection of the ideal nano-material depend on their complex interaction with the cellular environment, their progressive accumulation in live tissues and their inefficient degradation. Only compatible structural, chemical and optical properties of the ideal nanostructures may provide advantages in cell delivery. Considerable efforts have been directed towards surface modifications ¹², multivalent attachment of small molecules ¹³ and coating for minimizing such effects. These measures also favour in vivo distribution through diversified biological organs and effective tissue specific targeting. Though use of nanomaterials has been successful in in vitro cultured cells¹⁴⁻¹⁵, its in vivo application is more challenging for shelf life, potential immunogenicity, biocompatibility and other physiological hurdles.

### SUMMARY OF INVENTION

In one aspect, the invention comprises a nanostructure comprising a drug moiety or its derivative as a structural moiety that further has side chain/s capable of promoting self aggregation and/or performing a functionality, the side chains may be mono or a multiple of alkyl chain/s having even number or odd number of carbon atoms that may be substituted or unsubstituted for creating simple or complex assemblies.

In another aspect, the said drug moiety or its derivative is p-amino benzoic acid (PABA) derivative, a 4-alkylamido-N-pyridin-2-yl-benzamide of structure 1

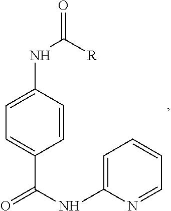

wherein the side chain R may be an alkyl group having carbon atoms 12 to 18 (C=12 to 18) or side chain/s capable of promoting self aggregation and/or performing a functionality, the side chains may be mono or a multiple of alkyl chains having even number or odd number of carbon atoms that may be substituted or unsubstituted for creating simple or complex assemblies.

The 4-alkylamido-N-pyridin-2-yl-benzamide of illustrated invention comprises a lauric (C=12) or stearic (C=18) side chain.

The nanostructure of the invention comprises nanotubes or nanorods wherein at least one structural moiety of the nanotube comprises a PABA derivative or a 4-alkylamido-N-pyridin-2-yl-benzamide of structure 1

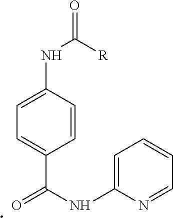

wherein the side chain R is as described above.

This invention comprises nanostructures wherein the said PABA derivative providing structural moiety comprises a derivative of 4-Amino-N-pyridine-2 yl-benzamide of structure 4

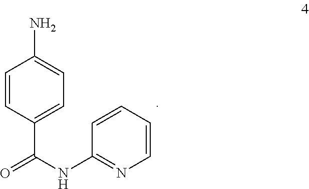 [SMILES: Nc1ccc(C(=O)Nc2ccccn2)cc1]

A nanotube or nanorod of this invention also comprises PABA as a linker for lauric (C=12) and/or stearic (C=18) side chains.

A nanotube or nanorod of this invention having a PABA structural moiety of a drug structural moiety further comprises conjugation of an active ingredient with the said nanotube or nanorod, wherein the active ingredient has a function to perform when absorbed or introduced in the body of a cell or an organism. The function may include, without limitation, a therapeutic action, in vivo tracking, or delivery of genetic material inside a cell or to an organism. In one particular embodiment of this invention the said conjugation is done with Rhodamine B as an active ingredient, which is useful for in vivo tracking. For the purpose of this specification, the term “conjugation” is used for a process or a mechanism that leads to formation of a single structure that is formed from association of nano-structures and an active ingredient/molecule by any mechanism that results into an associated structure and such mechanism may include entrapment, physical attraction forces, embedding, intercalation, covalent bond etc.

The invention also comprises a process of producing nanotubes or nanorods comprising structural moiety of a drug that has minimum obstacles of shelf life, potential immunogenicity, biocompatibility and other physiological hurdles in vivo application of nanomaterials and a side chain/s that facilitate self assembly and the said structural moiety comprises p-amino benzoic acid (PABA) moiety and the said side chain/s comprise/s a lauric (C=12) and/or stearic (C=18).

The process of this invention further comprises adding 4-Dodecanamido-N-(pyridine-2-yl)benzamide and N-(pyridin-2-yl)-4-stearamidobenzamide in a solvent and allowing the nanotubes or nanorods to get self assembled. In a particular embodiment the said solvent is methanol. In the process, equal weight of 4-Dodecanamido-N-(pyridine-2-yl)benzamide and N-(pyridin-2-yl)-4-stearamidobenzamide are heated with methanol about two times the weight of each of them, upon cooling to room temperature, deionized water in volume equal to the volume of methanol was mixed to obtain the final aggregates.

Another embodiment of this invention comprises a process of preparation of a 4-alkylamido-N-pyridin-2-yl-benzamide comprising the steps of (a) taking a mixture of 4-Amino-N-pyridine-2 yl-benzamide and pyridine in dry THF, (b) adding an acid chloride, (c) refluxing the mixture for overnight, (d) removing pyridine and THF under reduced pressure, (e) washing the residue with saturated NH₄Cl, (f) extracting with dichloro methane (DCM), (g) drying the organic layer over Na₂SO₄ and concentrating under reduced pressure, (h) PABA retaining its own functional properties in the self-assembled tubes.

A yet another embodiment of this invention of this invention, the said 4-alkylamido-N-pyridin-2yl-benzamide comprises 4-Dodecanamido-N-(pyridine-2-yl)benzamide and the said acid chloride used is Dodecanoyl chloride, or/and N-(pyridin-2-yl)-4-stearamidobenzamide is produced when the said acid chloride used is Octadecanoyl chloride.

In one aspect, this invention embodies a process of in vivo tracking by using a nanotube or nanorod of claim 6 with intrinsic fluorescence or by using a nanotube or nanorod conjugated with a fluorescent moiety. In one particular embodiment the said fluorescent moiety is Rhodamine B.

In a further embodiment, this invention comprises orally ingestible nanotubes or nanorods. The said nanotubes or nanorods serve the purpose of intracellular uptake and intercellular delivery of drugs or fluorescent molecule or tracking molecule or an active ingredient/s in cells of insects and human being.

Invention also embodies in nanotubes or nanorods that have at least one of the following properties: (a) green intrinsic fluorescence under confocal microscope, (b) red fluorescence when Rhodamine B is conjugated or any other fluorescence derived from the molecule conjugated, (c) randomly oriented structures of variable sizes that grow individually during the self-assembly process as seen under 3D Scanning Electron Microscopy images of nanostructures with as well as without Rhodamine or an active molecule, (d) nanotubes with variable sizes under Dynamic light scattering (DLS), the distribution starting with nano-size particles in fresh preparation to about micron size on prolonged storage up to several days, higher magnification images showing the hollow structures, (e) Rhodamine incorporated nanotubes having average heights of the majority of the nanotubes are 3-5 nm as seen in. 3D reconstituted AFM images, (f) soluble in DMSO and ethyl alcohol soluble, but insoluble in water, (g) can be stored in DMSO and an ethyl alcohol/water mixture for long periods without losing their properties during aggregation, (h) biocompatible with at least any one of the following: human embryonic kidney cells, neoplastic HeLa cells, Drosophila larvae, Drosophila adults, Drosophila eggs, Drosophila brain, Drosophila lumen or any other insect cells or human cells, (i) no adverse effect on mortality rates, physiology or locomotion of Drosophila on oral feeding or on external exposure to dry composition, (j) 200 nm in width and 500 nm in length to micron-sized structures in few aggregates, (k) efficient molecular transporters for different biologically important cells with no cytotoxicity, (l) side chain variations leading to changes in nanotube distribution in the body parts, shorter length of the lauric side chain exhibiting greater accumulation of nanostructures; the cells of different discs and larval brains being devoid of any nanotubes containing longer side chains (C=18), a considerable amount of nanotubes containing short chains (C=12) penetrating into the same tissues, (m) overcoming pharmacokinetic barriers and showing an efficient cellular uptake, (n) retention of the original properties of PABA in self-assembly nanotubes increasing PABA concentration in the intestine during oral ingestion, helping in delivery a multifunctional effect including convert by intestinal bacteria it into folic acids, and protecting against strokes, cardiovascular diseases and certain cancers, playing a dual role, directly as a delivery vehicle of therapeutic agents and indirectly by preventing different diseases as a therapeutic agent itself, (o) useful to deliver small regulatory RNA and DNA as therapeutic materials, (p) useful to optimize pill-like properties for orally ingested materials as micropills to deliver biomaterials for effective gene therapy and novel cargoes for molecular populations.

Invention also comprises a process of synthesis of 4-N-pyridin-2-yl-benzamide comprising steps of (a) converting p-nitro benzoic acid to amide formation with 2-aminopyridine, (b) followed by reduction of nitro functionality by using Pd/C in MeOH-1, 4 dioxane mixture (1:3) as a solvent for hydrogenation. The process further comprises steps of: (a) adding Oxalyl chloride and catalytic dimethyl formamide (DMF) to a suspension of p-nitro benzoic acid in dichloromethane (DCM) under nitrogen atmosphere at about 0° C. and stirring at room temperature until all solid material dissolve completely, occasionally release, (b) adding slowly triethyl amine at 0° C., after about 30 minutes of stirring, adding 2-Amino pyridine and stirred for overnight, filtering and washing white solid precipitated out with methanol, if required, recrystallizing in 70% acetic acid water to get a compound of structure 3

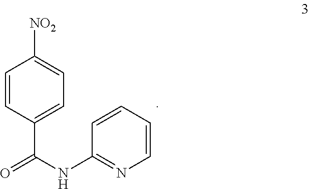 [SMILES: O=C(Nc1ccccn1)c1ccc([N+](=O)[O-])cc1]

This invention also comprises a process of claim 24 wherein 4-Amino-N-pyridine-2 yl-benzamide is synthesized comprising steps of: (a) slightly heating a suspension of 4-Nitro-N-pyridine-2 yl-benzamide in methanol and dioxane to form a clear solution, (b) carrying out hydrogenation for about 24 hours at 1 atmosphere pressure on a Pd/C (palladium on activated carbon) catalyst, (c) filtering the reaction through celite or any other filter aid, and (d) concentrating under reduced pressure to give a pale yellow solid of 4-Amino-N-pyridine-2 yl-benzamide, a compound of structure 4

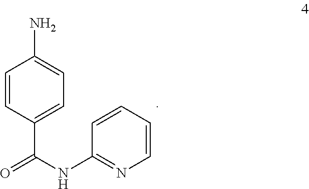 [SMILES: Nc1ccc(C(=O)Nc2ccccn2)cc1]

The invention comprises 4-alkylamido-N-pyridin-2-yl-benzamide of structure 1

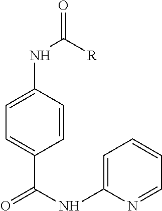

wherein the side chain R may be an alkyl group having carbon atoms 12 to 18 (C=12 to 18) or side chain/s capable of promoting self aggregation and/or performing a functionality, the side chains may be one or a multiple of alkyl groups having even number or odd number of carbon atoms that may be substituted or unsubstituted for creating simple or complex assemblies.

The invention also comprises 4-amino-N-pyridine-2-yl-benzamide of structure 4s

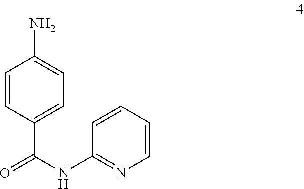 [SMILES: Nc1ccc(C(=O)Nc2ccccn2)cc1]

The invention also comprises N-(Pyridine-2-yl)-4-dodecanamido-benzamide of structure (1a):

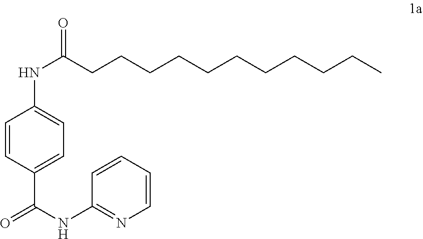 [SMILES: CCCCCCCCCCCC(=O)Nc1ccc(C(=O)Nc2ccccn2)cc1]

The invention further comprises N-(Pyridin-2-yl)-4-stearamidobenzamide of structure (1b):

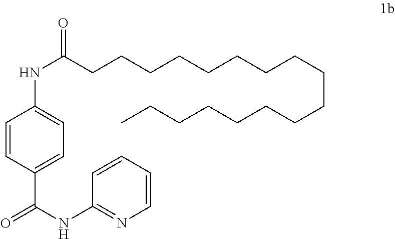 [SMILES: CCCCCCCCCCCCCCCCCC(=O)Nc1ccc(C(=O)Nc2ccccn2)cc1]

### DETAILED DESCRIPTION OF INVENTION

This invention embodies novel nanomaterials made from a commonly used structural moiety of a drug.

The invention for making nano-tubes from structural moiety of a drug is illustrated by use of p-aminobenzoic acid (PABA) that is frequently found as a structural moiety in different drugs. However, any other structural moiety of a drug that is easily absorbed in the body without triggering undesirable level of immunogenic response and further eliminated from the body without generating undesirable level of side effects may be used by using appropriate side chain/s that promote a self assembly to form nanostructures capable of being taken up by an organism after administration either for application for in vivo tracking or/and conjugating with an active as its carrier for administration to an organism for carrying out a certain function.

One embodiment of this invention comprises preparing nanostructures from p-amino benzoic acid. In another embodiment the invention, the said nanostructures comprise nanotubes, nanorodes, nanocube and all structural variations. In yet another embodiment, the invention comprises a process of preparation of nanotubes wherein PABA is used as building blocks for synthesis of nanostructures. In a further embodiment of the invention, the said nanostructures are used for oral administration. In an illustrative embodiment of this invention, two, 4-alkylamido-N-pyridin-2-yl-benzamides are synthesized using p-aminobenzoic acids (PABA) as a linker for lauric (C=12) and stearic (C=18) side chains. Any other side chains may replace lauric and stearic side chains that are capable of aligning/interacting with each other in a solution to result into self assembled nanostructures such as nanorods or nanotubes. Thus, although this invention is illustrated with (C=12) and (C=18) side chains, they may be replaced by even lower side chains than (C=12) and larger than (C=18). The side-chains can have either even number or odd number of CH2 groups in the side chain or may be even substituted and even can have multiple alkyl chains and in this way they enable creating functionality and for creating more complex assemblies. Thus, they may be replaced by even or odd numbered mono or multiple alkyl chains either substituted or not substituted. The structures may vary from small particles to long tubes depending on whether they are low carbon number side chains or large carbon number side chains. A further embodiment of this invention comprises nanotubes that emit fluorescence. In a still further embodiment, the said fluorescence may be an intrinsic fluorescence (green). A yet another embodiment of this invention comprises nanotubes that have standard fluorescence and non fluorescence marker dye embedded in tube walls. In an illustrative embodiment of this invention, the said standard fluorescence is derived from (Rhodamine B, red) embedded in the tube walls. Rhodamine B may also be replaced by any other fluorescent molecule capable of reacting with constituents of the nanotubes. Thus, this invention also comprises nanotubes and nanorods that are useful for their in vivo tracking in a live organism. In a still further embodiment of the invention, the nanomaterials emit fluorescence derived from the intrinsic as well as standard fluorescence and non fluorescence marker embedded in the tube walls. The invention comprises development of dual-purpose orally ingested carriers containing drug PABA structural moiety optimized for multi-dimensional biological applications.

The said tracking is demonstrated in Drosophila. Invention also comprises use of two side chains (C12 and C18) that are useful in preparing optimal nanotubes of PABA design for self assemble, oral delivery and biocompatibility.

The cellular uptake and intracellar delivery was also monitored in insect and human cultured cells. After incorporation, these organic nanostructures do not produce any adverse effect on physiology, behaviour, toxicity in Drosophila and growth of culture cells. This study provides the first step towards the development of dual-purpose carriers containing drug structural PABA moiety optimized for multi-dimensional biological applications.

### DETAILED DESCRIPTION OF INVENTION

To minimize the obstacles of shelf life, potential immunogenicity, biocompatibility and other physiological hurdles in vivo application of nanomaterials, a more reliable choice is to select compounds that are routinely used for structural moiety of different drugs¹⁶, as building block of nanomaterials.

To take such advantages in the preparation of nano-device, in this study we have selected p-aminobenzoic acid (PABA), frequently used as structural moiety of commercial oral drugs ¹⁷, as a linker for lauric and stearic acid side chain for making tube like nanostructure. PABA (p-aminobenzoic acid) is a part of folic acid and the vitamin-B complex that normally occurs in human liver. It is a natural non-protein amino acid which is frequently found as a structural moiety in drugs with a wide range of therapeutic applications, such as antibacterial, local anaesthetic, antiarrhythmic, gastrokinetic, etc [12]. PABA is also known to support folic acid production by intestinal bacteria[13]. These nanostructures minimize many obstacles including absorption in body fluids, systemic spreading and the reduction of immuno-genetic symptoms. To date, the potential use of drug components for synthesizing the microstructure has not been realized primarily because of lack of methods for self-assembly to form nanotube and coupling with tracking fluorescence markers. To trace the nano-device through the complex biological system, in depth fluorescence imaging was performed. The tubes also maintain their fluorescent activity for a long period of time and do not suffer from dissociation or decay of fluorescence. In nanotubes p-aminobenzoic acid is coupled with stearic acid side chain that emits intrinsic fluorescence or lauric acid with Rhodamine B, embedded on the wall.

These organic tubes were further tested for biocompatibility and bioavailability to sensitive internal organs of Drosophila thereby sets new parameters to compliment the in vivo studies.

Following are examples that illustrate how the invention works. The examples are only illustrative of the invention, and do not limit the scope of the claims, that may cover equivalents which may not be explicitly covered here but provide the same results as provided by following examples, and are obvious to a person of ordinary skill in this art.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1. Chemical synthesis, structure and physical properties of p-aminobenzoic containing nanotubes. a) Chemical synthesis of compound 1a, 1b; b) Schematic diagram of self assembled compound 1a, 1b forming PNT-A and PNT-B; c) structure and different microscopic views (Confocal fluorescence Microscope, AFM, SEM) are shown. Enlarged view of a single microsized tube (1-3 μm) is shown as an insert in each figure. Scale-3 μm (Confocal and SEM) and 250 nm (AFM).

FIG. 2. ¹³C NMR spectra of compound 4-Dodecanamido-N-(pyridine-2-yl)benzamide(1a).

FIG. 3. ¹H NMR spectra of compound 4-Dodecanamido-N-(pyridine-2-yl)benzamide (1a)

FIG. 4. ¹³C NMR spectra of compound N-(pyridin-2-yl)-4-stearamidobenzamide (1b).

FIG. 5. ¹H NMR spectra of compound N-(pyridin-2-yl)-4-stearamidobenzamide (1b).

FIG. 6. Incorporation of PNT-A nanotubes in tumor HeLa cells after incubating 48 hrs in nanotube and DMSO (0.1%) containing media. The merge figure showed the localization of nanotubes in the cells. Scale-25 μm

FIG. 7 Biocompatibility of nanotubes in insect Drosophila S2, nonneoplastic (HEK-293) Human cells (a, b) Specificity of cellular uptake of two different (PNT-A, PNT-B) nanotubes in insect (Drosophila S2) (a) and Human Embryonic Kidney (HEK-293) (b) cultured in 0.1% DMSO and 60 μg/ml nanotube containing media for 12 hrs cells. Scale −25 μm. (c) Proliferation of insect S2 and human (HEK-293) cells, carried out after 1-3 days of incubation in normal media, media containing 0.1% DMSO or PNT-B nanotubes (40 μg/ml and 60 μg/ml). The absorbance in 570 nm is linearly dependent on the number of viable cells (d) Cytotoxicity assay for the same cell lines incubated 24 hrs at various concentrations nanotubes was performed. The same cells were treated to a fixed concentration of DMSO (0.1%) as internal control. Cytotoxicity values correspond to the percentage of dead cells.

FIG. 8 Viability of different life stages of nanotube fed Drosophila. The comparison of survival rates in egg, larva, pupa and adult stages of Drosophila were estimated after feeding the entire larval period exclusively on yeast paste containing nanotubes and DMSO (0.1%). Each group contains 10 batches of 180 first instar larvae. The average percentages of survivals in each stage were plotted in bar diagram.

FIG. 9. Biocompatibility assay of PNT-A and PNT-B tubes after oral ingestion in live Drosophila
a) Viability of nanotube and DMSO fed larvae, pupae and adult flies were compared after feeding exclusively on yeast paste containing nanotubes and DMSO
b) The male/female ratios of emerged adults from the nanotubes (60 ug/ml), DMSO (0.1% and 10%) and normal paste fed population (control) were counted. The number of viable males and females were plotted in a bar chart
c) The effect of nanotubes feeding on Drosophila sex and female egg laying capacity were estimated. Mean daily fecundity (egg laying) per female of nanotubes or DMSO fed population of female Drosophila consecutive six days after hatching was calculated.
d) Long sustainability and shelf life of organic nanotubes in Drosophila was estimated by comparing fluorescence images of adult Drosophila cultured with normal culture media for consecutive 7 days after hatching from the DMSO and PNT-A and PNT-B fed larvae.

FIG. 10. Distribution of fluorescence dye in different internal organs of DMSO fed larvae from different confocal microscopic images. The area is each organ was marked with white boarders.

FIG. 11. Distribution and spreading of two separate nanotubes in different organs of Drosophila larvae after oral ingestion; mean ratios of the fluorescent intensity from three independent values were plotted in a bar diagram.

FIG. 12. (a) Distinct distribution patterns of PNT-A and PNT-B in different external organs of adult flies. The intensity of fluorescence is proportional to the accumulated nanotubes inside the cells;

(b) bio-distribution and accumulation of two separate nanotubes in leg and wing discs and larval brains are markedly different.

### SYNTHESIS OF PABA-NANOTUBE

Initially, we have synthesized 4-N-pyridin-2-yl-benzamides from p-nitro benzoic acid. The synthesis involved amide formation with 2-aminopyridine followed by reduction of nitro functionality by using a modified protocol where Pd/C in MeOH-1,4-dioxane mixture (1:3) was used as a solvent for hydrogenation. The details are given in the following Scheme:

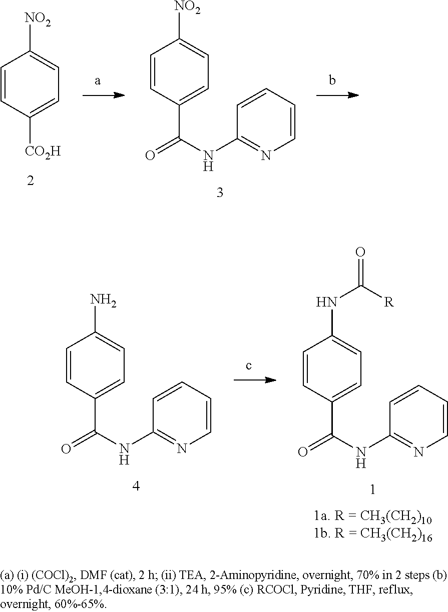

### Preparation of 4-nitro-N-pyridine-2-yl-benzamide (3)

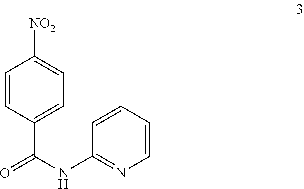 [SMILES: O=C(Nc1ccccn1)c1ccc([N+](=O)[O-])cc1]

Oxalyl chloride (5.68 mL, 65.8 mmol) and catalytic DMF (dimethyl formamide) (0.5 mL) was added to a suspension of p-nitrobenzoic acid (10.0 g, 59.8 mmol) in CH₂Cl₂ (100 mL) under nitrogen atmosphere at 0° C. and stirred at room temperature until all solid material dissolved completely. During this process gas evolves which was occasionally released. Triethyl amine (24.48 mL, 179.4 mmol) was slowly added at 0° C. and solution turned dark red. After 30 min of stirring, 2-amino pyridine (6.19 g, 65.8 mmol) was added and stirred for overnight. White solid precipitated out which was filtered and then washed with methanol (100 mL). The solid was re-crystallized in 70% acetic acid in water (400 mL) to furnish 3 (10.0 g, 70%) as a white solid.

### Preparation of 4-amino-N-pyridine-2-yl-benzamide (4)

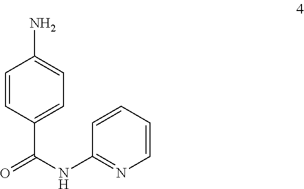 [SMILES: Nc1ccc(C(=O)Nc2ccccn2)cc1]

The suspension of 4-nitro-N-pyridine-2-yl-benzamide (3) (5.0 g, 20.5 mmol) in a mixture of MeOH (15 mL) and of 1,4-dioxane (45 mL) was slightly heated to form a clear solution. 3.5 g of 10% Pd/C (palladium on activated carbon) was charged and hydrogenation was carried out for 24 h at 1 atm pressure. The black reaction mass was filtered through Celite bed and then concentrated under reduced pressure to produce 4 (4.05 g, 95%) as a pale yellow solid. The solid material was used for next reaction without further purification.

### General Procedure for Preparation of Compounds (1a and 1b)

To a mixture of 4-amino-N-pyridine-2-yl-benzamide (4) (0.5 g, 2.34 mmol) and pyridine (2 mL) in dry THF (15 mL), was added respective acid chlorides (2 equivalents) (acid chlorides of lauric acid or stearic acid). The resultant mixture was refluxed for overnight and then pyridine and THF was removed under reduced pressure. The residue was washed with saturated NH₄Cl (2×20 mL) and extracted with CH₂Cl₂ (dichloromethane). The organic layer was dried over Na₂SO₄ and concentrated under reduced pressure. Purification by silica gel column chromatography (20% EtOAc/hexane) produced compound N-(pyridin-2-yl)-4-stearamidobenzamide (1a) (0.7 g, 62.4%) and compound N-(pyridine-2-yl)-4-dodecanamido-benzamide (1b) (0.6 g, 64%) as a white solid.

### Characterization of Compounds

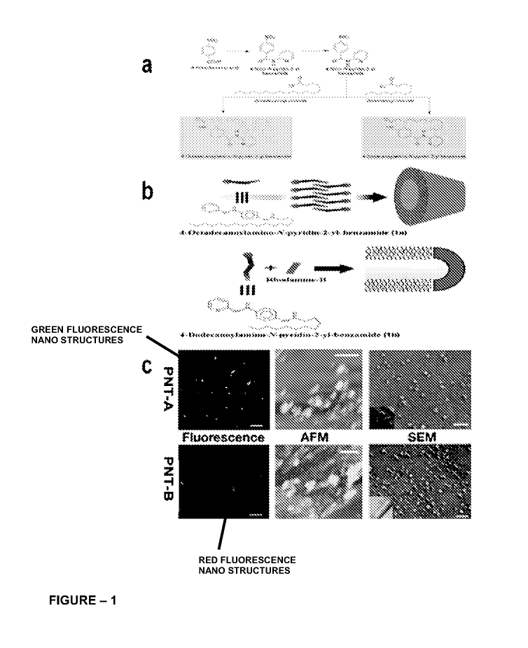

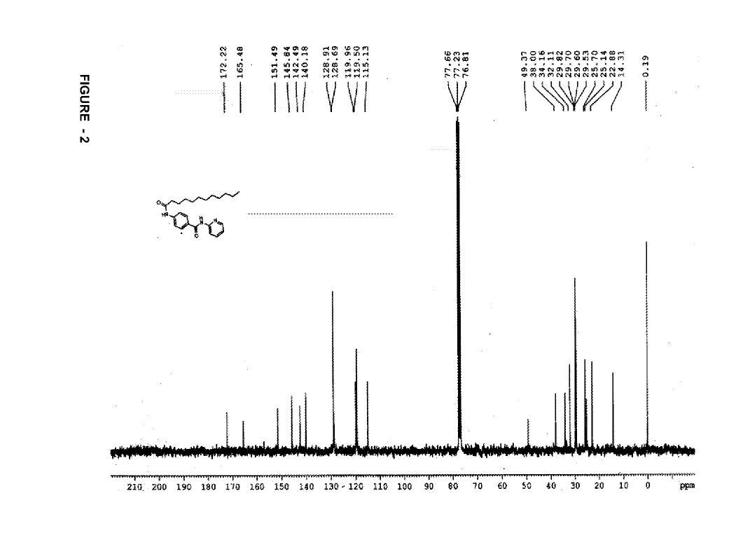

FTIR studies were performed to see the hydrogen bonding and their role in assemblies. Scanning electron microscopy (SEM), transmission electron microscopy (TEM) and confocal microscopy were used to examine the self-assembled fluorescent molecules (FIG. 1(b)). Analysis by laser confocal microscopy revealed that PNT-A showed green intrinsic fluorescence and PNT-B with rhodamine B showed red fluorescence. The 3D SEM images showed that PNT-A and PNT-B are randomly oriented structures of variable sizes that grow individually during the self-assembly process. Dynamic light scattering (DLS) studies also showed nanotubes with variable sizes. The distribution becomes broader over time, indicating a dynamic process of assembly. Initial imaging of a few batches of nanoparticles collected from a fresh preparation are distinct from images of the same particles after storage in a methanol and water mixture for three consecutive days. The images were inspected from several batches of nanoparticles collected at different time points of storage. The nanoparticles from the fresh preparation were mostly nano in size with uniform shape, but prolonged storage (several days after PNT was stored in solution) tended to form sub-micron-sized tubes. Higher magnification images confirm the hollow structures. The structure of the PNT-B is hollow even after fluorescent molecules are added. 3D reconstituted AFM images showed the average heights of the majority of the nanotubes are 3-5 nm only (FIG. 1(b)). Both nanomaterials can be stored in DMSO (0.01%) and an ethyl alcohol/water mixture (1:2 v/v) for long periods without losing their properties during aggregation. To ascertain whether a mixture contains intact nanotubes, several SEM samples were prepared and processed from the same ethyl alcohol/water mixture. SEM images showed a morphology without any sign of degradation.

The mechanism and the driving force for the formation of nanometric morphologies are not clearly understood. As the two compounds possess a pyridine, benzene and alkyl chain, it is likely that hydrogen bonding, π-π interactions and vander Waals interactions play a major role in directing the aggregation and subsequent nanotube formation. The FTIR study revealed two absorption bands of anti-symmetric (vₐₛ) and symmetric (vₛ) CH₂ stretching vibrational modes at 2920 cm⁻¹ and 2850 cm⁻¹, which confirmed the presence of a highly ordered trans structure in the aliphatic region. The absence of an amide A band, attributed to a free NHgroup (over 3430 cm⁻¹), and the presence of an N—H stretching frequency near 3310 cm⁻¹ further indicates that all NH-groups are involved in an extensive hydrogen bonding network[15]. The CO stretching band at around 1600 and 1659 cm⁻¹ (amide I) and the NH bending peak near 1517 cm⁻¹ (amide II) suggest β-sheets for the molecules in the solid state[16], which are known to form weakly curved sheets. The presence of sheets, half-tubes and nanotubes gives evidence for the ‘rolling’ mechanism [17]. The formation of the tubular structures may occur by a closure of the extended sheet along one axis of the two-dimensional layer (FIG. 1(c)).

To verify the fluorescence enhancement induced by self-assembly of PNT-A, the fluorescence emission of the monomer and PNT-A was studied using a Nano drop 3300 fluoro-spectrometer. The fluorescence intensity of the PNT-A (in methanol/water solution) using the blue diode option (maximum excitation 477 nm) was very strong and found at 510 nm in contrast to that of the no-fluorescent monomer (studied in CH₂Cl₂ where it does not aggregate) under the same concentration of 0.3 wt %. The self-assembly occurring may be due to the stack arrangement of molecules and the transition to the lower couple excited state of the molecules favoured the enhancement of the emission. Compound PNT-B conjugated with a shorter lauric acid side chain (C=12) did not emit intrinsic green fluorescence.

### N-(Pyridine-2-yl)-4-dodecanamido-benzamide (1a)

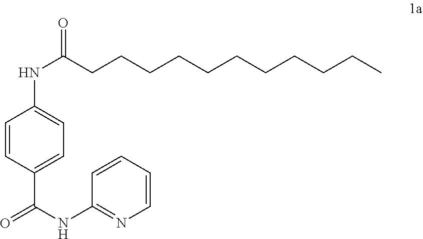 [SMILES: CCCCCCCCCCCC(=O)Nc1ccc(C(=O)Nc2ccccn2)cc1]

White solid; M.p: 152-154° C.

IR (neat): 3384, 3319, 2920, 2849, 1648, 1587, 1516, 1310, 1246, 1178, 831, 777, 676, 637 cm⁻¹; ¹H NMR (200 MHz, CDCl₃+DMSO-d₆) δ 12.05 (S, 1H), 9.64 (S, 1H), 8.90 (d, J=8.5 Hz, 1H), 8.37 (d, J=5.4 Hz, 1H), 8.27 (d, J=8.5 Hz, 2H), 8.22-8.15 (m, 1H), 7.84 (d, J=8.5 Hz, 2H), 7.38 (t, J=6.2 Hz, 1H), 2.41 (t, J=6.9 Hz, 2H), 1.79-1.64 (m, 2H), 1.38-1.22 (m, 18H), 0.88 (t, J=6.9, Hz, 3H); ¹³C NMR (300 MHz, CDCl₃) δ 172.22, 165.48, 151.49, 145.84, 142.49, 140.18, 128.91, 128.69, 119.96, 119.50, 115.13, 49.37, 38.00, 34.16, 32.11, 29.82, 29.70, 29.60, 29.53, 25.70, 25.14, 22.88, 14.31.

HRMS for C₂₄H₃₄O₂ calculated 396.2651. found 396.2645.

### N-(Pyridin-2-yl)-4-stearamidobenzamide (1b)

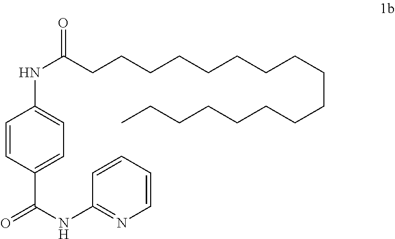 [SMILES: CCCCCCCCCCCCCCCCCC(=O)Nc1ccc(C(=O)Nc2ccccn2)cc1]

White solid; M.p: 140-142° C.

IR (neat): 3312, 2921, 2852, 1745, 1659, 1591, 1518, 1432, 1315, 1245, 1177, 854, 775, 684, 647 cm⁻¹; ¹H NMR (200 MHz, CDCl₃+DMSO-d₆) δ 10.30 (S, 1H), 9.69 (S, 1H), 8.44 (d, J=8.6 Hz, 1H), 8.30 (d, J=4.6 Hz, 1H), 8.01 (d, J=8.6 Hz, 2H), 7.92-7.79 (m, 1H), 7.75 (d, J=9.3 Hz, 2H), 7.12 (t, J=6.2 Hz, 1H), 2.34 (t, J=7.81 Hz, 2H), 1.68-1.61 (m, 2H), 1.27-1.19 (m, 28H), 0.83 (t, J=6.2, Hz, 3H); ¹³C NMR (300 MHz, CDCl₃+DMSO-d₆) δ 172.13, 165.23, 151.34, 146.23, 142.60, 138.47, 128.17, 127.64, 119.11, 118.57, 114.30, 36.86, 31.33, 29.10, 28.96, 28.92, 28.77, 25.05, 22.11, 13.63.

HRMS for C₃₀H₄₆N₃O₂ calculated 480.3590. found 480.3587.

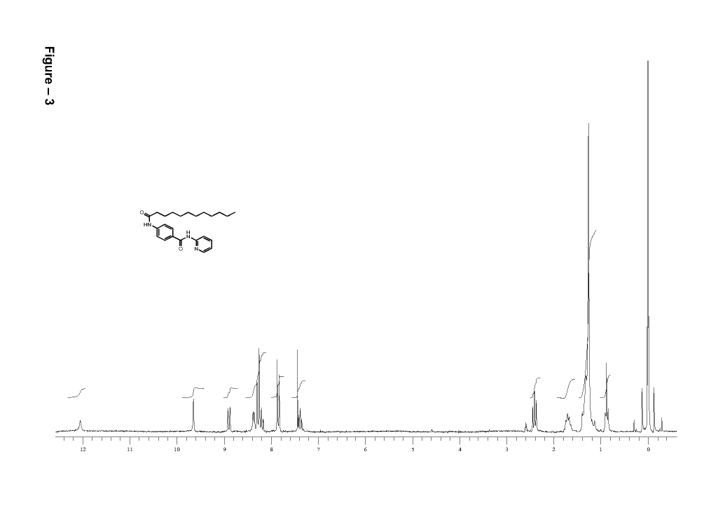

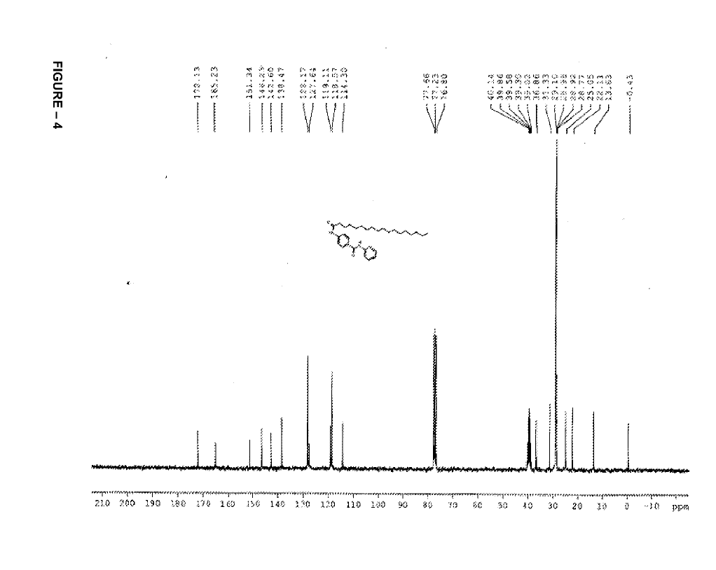

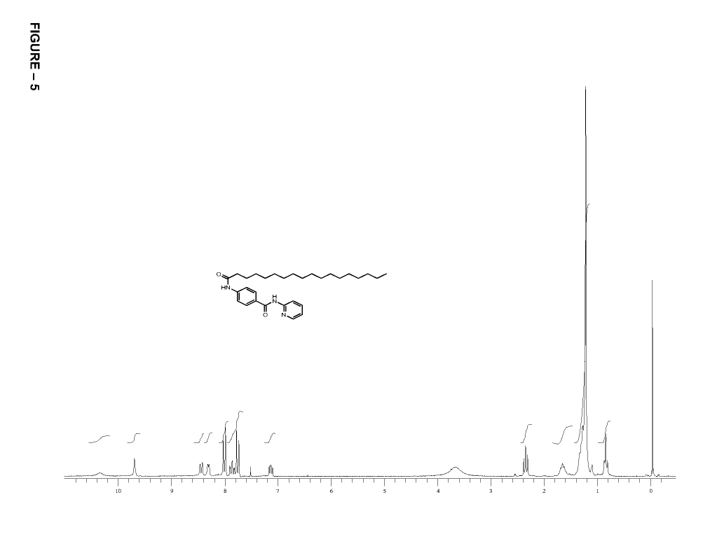

The NMR and ¹³C Spectra are shown in FIG. 2 to FIG. 5

Subsequently, the benzamides were coupled with two acid chlorides (Octadecanoyl chloride, C=18 or Dodecanoyl chloride, C=12) to furnish two 4-alkylamido-N-pyridin-2-yl-benzamides (compounds 1a and 1b) (FIG. 1a). Self-assembly of compound 1a and 1b was performed by heating 1 mg of each of the compound in 2 ml of methanol till it dissolved completely. Upon cooling to room temperature, 2 ml of deionized water was mixed to obtain the final aggregates.

To obtain visual images of assemblies including 3D structure and measurements, several images from fluorescent laser confocal microscope, Atomic force microscope (AFM) and scanning electron microscope (SEM) were analyzed (FIG. 1c). The 3D images showed that self-assembled benzamide nanomaterials (PNT-A and PNT-B) form tubes of variable size with space inside. Tubes are mainly less than 200 nm in width and 500 nm in length, but in few aggregates they formed micron-sized structures. The PNT-A showed green fluorescence colour that emits from self-assembled 4-alkylamido-N-pyridin-2-yl-benzamides coupled with a stearic side chain (C=18). Compound PNT-B conjugated with a shorter lauric side chain (C=12) does not emit intrinsic fluorescence. To add standard fluorescence, PNT-B organic tubular structure was synthesized by mixing Rhodamine B with tube-forming benzamide during the self-assembly process. The Rhodamine B that is embedded in the wall of self-assembled nanotubes (PNT-B) emits red fluorescence as specific signal. Both nanomaterials are DMSO and ethyl alcohol soluble, but not in water and can be stored for long period without losing its properties. To investigate whether after conjugation with acid chloride, p-amino-benzoic acid retains its own biological properties in the self-assembled tubes, growth and viability of the wild type bacterial stains (E coli K12) that are cultured in a normal media in the presence of PABA or PABA containing microstructure was estimated. The identical growth pattern of bacteria in two separate cultures for 7 consecutive days revealed that PABA retains absolutely its own functional properties in the self-assembled tubes.

### Biocompatibility in Insect, Human Embryonic Kidney and Neoplastic Hela Cells

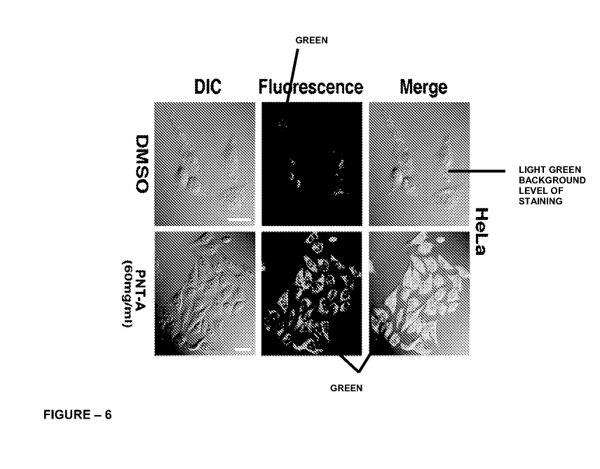

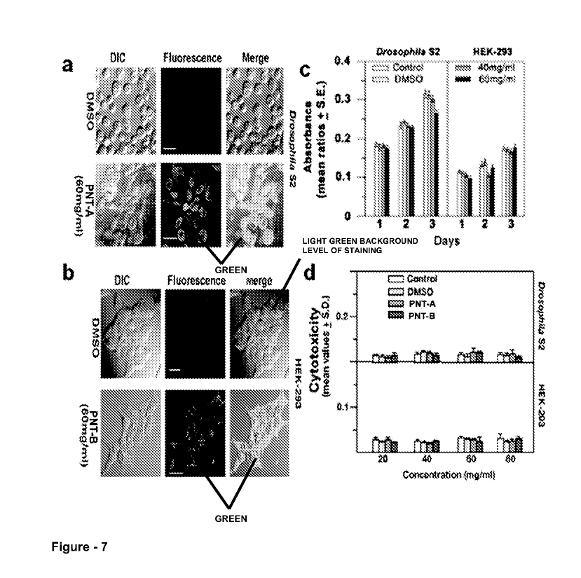

Next, insect and mammalian cells were used to estimate the efficiency of cellular uptake of two nanotubes in vitro. Drosophila S2 cells, nonneoplastic Human Embryonic Kidney (HEK-293) and neoplastic HeLa cells were grown in small dishes or cover glasses and incubated with the nanostructures dissolved in 0.1% DMSO that has no adverse effect on cell physiology¹⁴. After 24 or 48 hrs incubations, cells were fixed in 4% paraformaldehyde, followed by few gentle washes with PBS. The cells were viewed under laser confocal microscope (Olympus FV1000). The reconstituted images showed that both PNT-A and PNT-B was accumulated in the periphery of the nucleus of both insect and human (HEK293, HeLa) cells incubated with 60 ug/ml nanotubes and 0.1% DMSO (FIGS. 6, 7a and 7b). The accumulation was increased proportionately to the concentration of nanotubes in the media. In contrast, a negligible amount of fluorescence signals was emitted from the cells incubated in 0.1% DMSO alone for the same period of time. These findings demonstrate that nanostructures are accumulated in the cultured cell after penetrating the plasma membrane. Moreover, an identical distribution pattern of fluorescence emitted from PNT-A and PNT-B in the insect and human cells further verified that similar to intrinsic green in PNT-A, embedded red fluorophore remains coupled with PNT-B tube wall after accumulation in the cytosol.

To test the viability of the cultured cells, a colorimetric assay was performed using 3-(4-5-dimethylthiazol-2-yl)-2,5-diphenyltetrazolium bromide as described earlier ²¹. This assay is based on the reductive capacity to metabolize the tetrazolium salt to blue coloured formazone. Cells were incubated with various concentrations of nanotubes. After incubation, cells were added with MTT and the absorbance of coloured product was monitored by a microplate reader at 570 nm. In all the cases tested, the cell proliferation estimated by the absorbance demonstrates that incorporation of nanotubes does not influence cell proliferation. Further the viability of Drosophila S2 and noncancerous HEK-293 cells in the presence of nanotubes showed more than 95% viability relative to DMSO treated cells (FIG. 7c, 7d). These results suggest that nanomaterials are the efficient molecular transporters for different biologically important cells with no cytotoxicity.

### In Vivo Tracking of Nanotubes in Drosophila

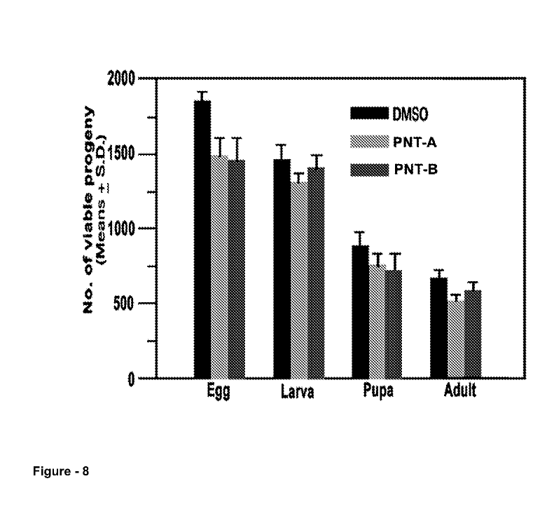

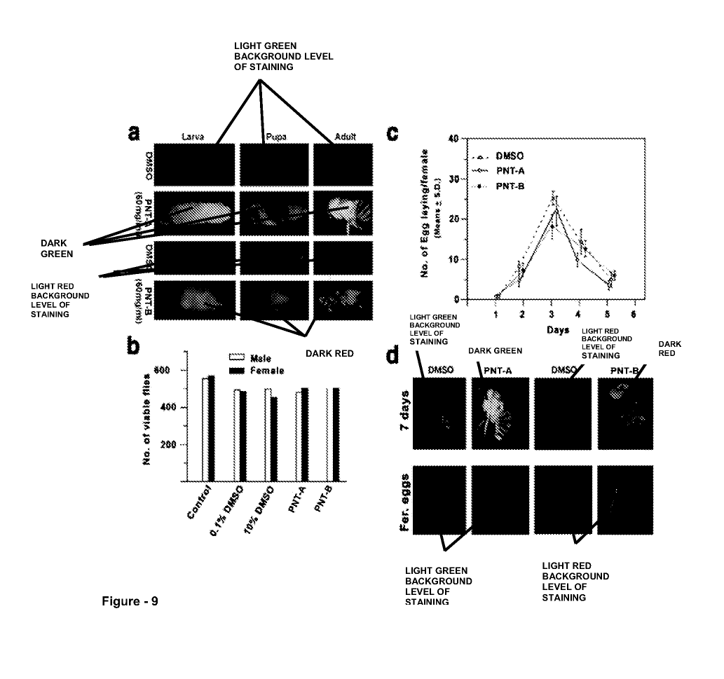

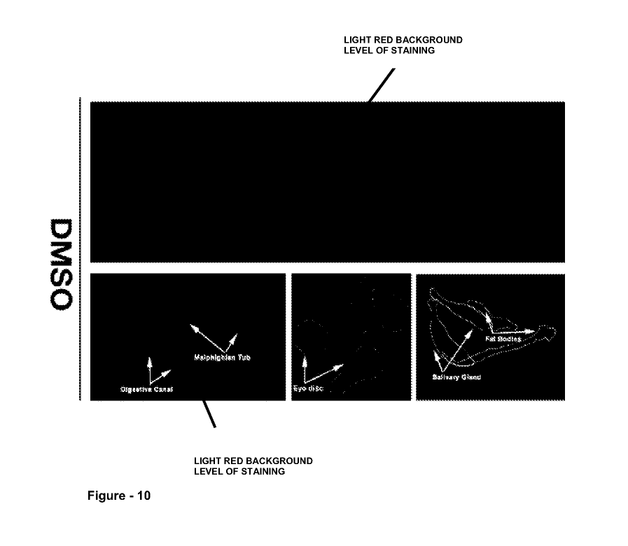

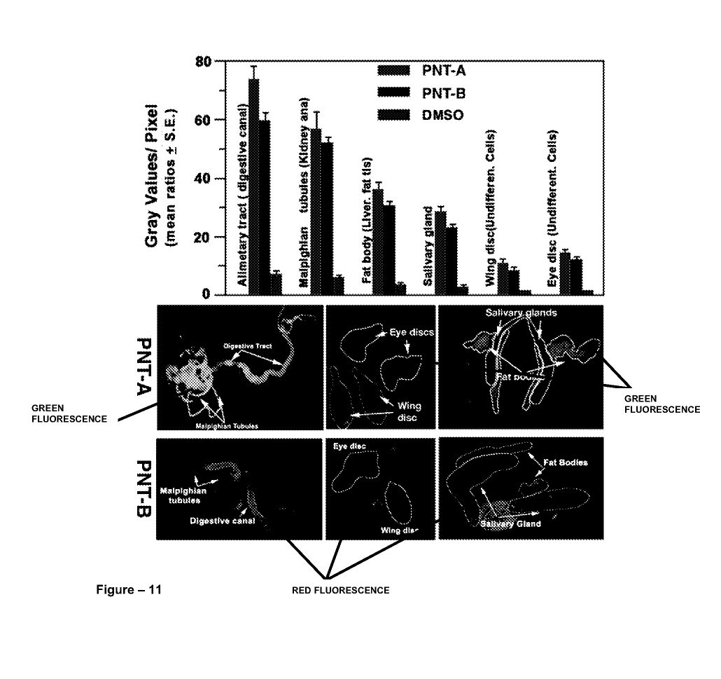

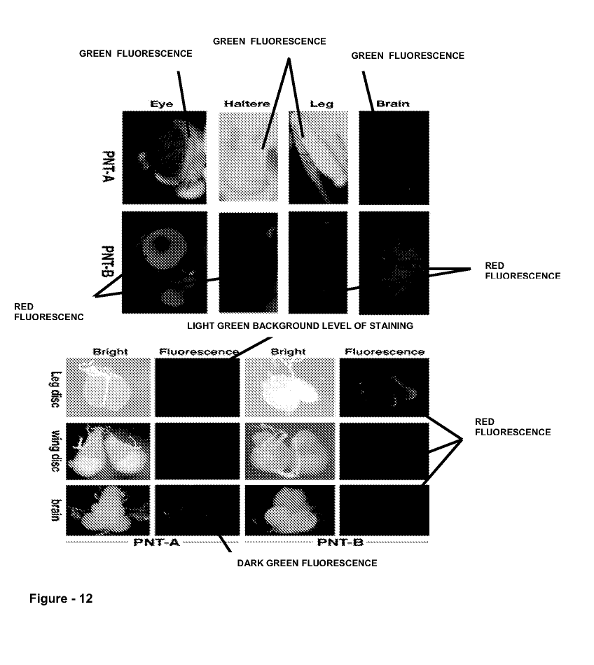

These nanosized structures were further scaled up for in vivo use precisely to facilitate organ distribution and to combat different physiological hurdles in the live organisms. To test overall viability and growth, after feeding the PABA containing nanotubes to first Instar larvae, development of mature larvae, pupae and adult Drosophila were estimated (FIG. 8). After hatching, larvae undergo an intense 4-5 day feeding, when they increase weight by 200-folds. In the subsequent immobile pupal stage, they stop feeding; therefore no marked gain in adult flyweight from the larval stage was noticed. To feed larvae with highest possible doses of PNT-A and PNT-B, dry Baker's yeast was mixed with concentrated suspensions of nanostructures (60 μg in 100 μl) in 0.1% DMSO solutions, followed by centrifugation and decantation. The resulting pastes were used in equal amount as the sole food source for various batches of equal number of larvae. Indeed viability of the larvae, pupae and adult was marginally higher in sole DMSO (0.1%) fed flies relative to the flies fed on yeast paste containing nanotubes (FIG. 10a). The feeding of low percentage DMSO (0.1%) has no apparent effect on fly physiology and viability (FIG. 9).

To investigate the effect of the nanotubes on overall growth, behaviour and physiology of the flies, the size of the adults and the sexual behaviour of newly emerged flies were monitored. No size difference between DMSO and PNT-A and PNT-B fed male and female flies and abnormalities in their sexual behaviour were marked (FIG. 10b). The egg laying capacity of adult females for six consecutive days after hatching and the sex of enclosed flies were counted. No significant difference was detected in egg laying capacity and male/female ratios when compared to the wild type, DMSO or nanotubes fed females (FIG. 10b, 10c). Taken together, these organic nanotubes have no adverse effect on the adult flies, generated from feeding nanotubes compared to DMSO-fed first instar on cellular physiology, behaviour, f fly sex and other pharmacokinetics parameters.

Next, we demonstrated the organ-selective distribution of body parts of the nanotube-fed flies reared in normal food nanotubes and their clearance from live organisms after sole media for 0-7 days. Thus Drosophila cultured in normal media without nanotubes does not reduce the stability and shelf life of the fluorescent organic nanotubes for a limited period. The fluorescence intensity was reduced conspicuously after extending the culture on an average of 23-25 days and was nearly eliminated from the organs of the adult flies reared in a 47 days-old culture. We further examined fluorescence intensity of the mature fertilized eggs laid by nanotube-fed females reared in normal food for 7 days after hatching. No fluorescence signal was emitted from the eggs. It eliminates the rare possibility of genetic inheritance of nanomaterials in successive generations through germ cells. Therefore, lack of heritable transfer of nanotubes leads to an ineffective route of nanotube spreading in the environment and their natural entry into the food chain via participating eco-consumers.

Earlier studies have shown dry exposure of carbon nanotubes in adult Drosophila. Materials adhering to the body surface of flies led to impairment of natural and rapid locomotion resulting in ultimate death [19]. To ensure that the exposure of these PABA nanotubes to adult flies does not promote any locomotory problem or environmental hazards, the ability of adult Drosophila to climb on the wall of 6 cm glass test tubes in the presence or absence of ample dry PABA nanotubes (PNT-A and PNT-B) was recorded using digital videography. The average height covered by each set of flies in the presence of two separate nanotubes as well as in the absence of nanotubes was measured and plotted in a bar diagram. Flies were allowed to reach the top of the glass tubes after being knocked into nanotube powder at the bottom of the test tubes. Unlike the effect of carbon nanotubes, exposure to PABA nanotubes does not prevent climbing as well as immobilization of flies in the bottom of glass tubes, as compared to the flies kept in the absence of PABA. Studies of locomotion indicated that secretion of footpads of Drosophila was crucial in adhering to a plain surface. If nanoscale materials attached to the fine pads on the fly foot or block the adhering fluids, they could limit the force required for climbing on a smooth surface. PABA nanostructures do not interfere with such events.

### Distribution in Internal Organs

In live insects, majority of the internal organs are submerged in haemolymph—a blood equivalent of human. The haemolymph circulates through the open vessel and pumps the fluid in a fixed direction, at the posterior body cavity by using a series of valves that prevent opposite haemolymph flow. As reported earlier by feeding single walled carbon nanotubes in intact fly postulated that the fluorescent methods are ideal for delivery and diagnostic application ⁵. Feeding of nanotubes to larvae and adults causes systemic spreading of signals by the gut peristaltic movement to cross the cell membrane barrier. The intensity of the dye associated with the nanosized materials is proportionate to the amount of accumulated tubes that were incorporated in the gut cells ^{22,23}. Variable intensity of fluorescence in the different parts of the body demonstrates different amount of nanotubes accumulation in the different organs. Internal organs of larvae and adult tissues were dissected, fixed in 4% paraformaldehyde, processed and scanned under Confocal Fluorescence Microscope. At least five samples of each organ from larvae fed on nanotubes were viewed and intensity of fluorescence scored. The amount of fluorescence of each tissue of the DMSO and nanotube fed larvae and adults was estimated (Gray value/pixel) using Metamorph software ²²⁻²³ An increased accumulation of fluorescence was found in the digestive track (83.06±4.51 in PNT-A), Malpighian tubules (57.19±3.81), fat bodies (38.41±2.19) compared to the salivary glands (27.73±1.81) and rapidly dividing cells of two imaginal discs (11.52±0.56 in wing discs and 14.32±0.41 in eye discs) (FIG. 8).

Next, to verify whether the physiological setting and pH of the body fluid has no impact on the degradation of the self-assembled nanotubes, we mixed rhodamine-B dye with yeast paste and served it as the sole food source of a few batches of first instar larvae. After feeding raw rhodamine-B the internal organs were dissected from the mature third instar larvae fed on the same food source. The rhodamine B is only accumulated in the gut cells after absorption, but the spreading of the red dye is nominal in all other organs examined. These results demonstrate that, similar to stem cells, the intensity of the red dye is emitted from intact nanotubes. Alternatively, raw rhodamine-B has no capability to enter into the complex and cell-dense internal organs of the larvae.

However, changes in size and surface textures associated with side chain variations in PNT-A and PNT-B lead to conspicuous changes in nanotube distribution in the body parts. The shorter length of the lauric side chain exhibited greater accumulation of nanostructures. The cells of different discs and larval brains were devoid of any PNT-A tubes containing longer side chains (C=18) but a considerable amount of PNT-B (C=12) penetrated into the same tissues. In adults, a distinct pattern of PNT-A and PNT-B distribution indifferent organs was found. PNT-A mainly accumulated in the abdomen and thorax including halters and legs as summing their prolonged retention in the fluid of the main body cavity, while PNTB conjugated with C-12 side chain was widely distributed in eyes, antennae, proboscis and adult brains (FIG. 12(a)). Therefore, side chain selection for organic nanotubes and cell physiology has profound effects on bio-distribution and clearance in vivo in different internal organs including leg and wing precursor cells. To examine the preferential penetration of two compounds carrying variable sidechains in adult brains, the amount of fluorescence in the brain tissues was analysed. The PNT-B with a short side chain has a clear advantage in the entry of the brain tissues over PNT-A in an equal concentration (FIG. 12(b)).

Interestingly, no change in fly behaviour after oral digestion suggests that accumulation of PABA nanoparticles does not show any permanent damage in the brain and other live organs because p-aminobenzoic acid functions in the improvement of neuro-degerative damages by inhibiting acetylcholinesterase ¹⁶. In particular, size and shape variations of nanotubes and their interactions in physiological settings are of paramount importance for their in vivo localization.

Further high concentrations of PNT-A were observed in murine stem cells and in the organs of Drosophila after feeding. An enlarged view of a mouse embryonic stem cell and leg disc (FIG. 12(c)) showed a dense accumulation of small tube-like impressions in the cytoplasm of stem cells and in fixed tissues. These findings allowed us to anticipate the accumulation of tube-like structures in two cell types. Our results demonstrate that differential uptake and specificity of nanomaterials in the live cells depend on the cellular physiology and chemical modifications of transporters.

Interestingly, no change in fly behaviour after oral digestion suggest that accumulation of PABA nanostructure do not produce any permanent damage in the brain because p-aminobenzoic acid functions in the improvement of neuro-degerative damages by inhibiting acetylcholinesterase ¹⁶.

Here, several critical questions relevant to biocompatibility and application of p-aminobenzoic acid containing nanostructures are addressed. We have determined the parameters of chemical, structural and biological modifications suitable for delivery in multipotent stem cells. It is also evident that distribution and accumulation is sensitive to the shorter (C=12) alkyl side chain. These organic nanotubes overcome pharmacokinetic barriers and show an efficient cellular uptake [15] even in biologically vulnerable and sensitive embryonic stem cells. Finally, modifications in alkyl side-chains play an important role in tissue and organ selective bio-distribution. The side chain modifications in PNT-A and PNT-B alter the shape of the nanostructures by changing self-assembly properties and surface coating, which leads to a distinct tissue-specific distribution specifically in adult eyes, its precursor cells, imaginal discs and neuronal tissues in larval and adult brains. The chemical modifications of the self-assembled p-aminobenzoic moiety also show a better stability and longer shelf life before degradation. But no short-term toxicity or impaired growth of Drosophila larvae and adults after feeding solely on nanotube-containing media was found.

On the other hand, administration of two different nanomaterials to adult flies elicits no response to locomotion and mortality. Therefore, exposure of PABA nanotubes did not produce any health and environmental hazards and do not permit fly-to-fly transmission. These results suggest that use of PABA nanotubes for mass delivery is safe and does not produce any adverse effect in nature. Further, the retention of the original properties of PABA in self-assembly nanotubes seems to increase PABA concentration in the intestine during oral ingestion, which might help intestinal bacteria to convert it into folic acids, and protect against strokes, cardiovascular diseases and even certain cancers. Therefore ingestion of PABA nanotubes plays a dual role, directly as a delivery vehicle of therapeutic agents and indirectly by preventing different diseases as a therapeutic agent itself [21]. This approach could be useful not only to deliver small regulatory RNA and DNA as therapeutic materials but also to optimize pill-like properties for orally ingested materials. Such attempts may favour making the next-generation micropills to deliver biomaterials for effective gene therapy and novel cargoes for molecular populations for the improvement of animal husbandry. Though the potentiality of PABA nanomaterials is advantageous over other delivery vehicles, it is still sensitive to cell types and physiology. On the other hand, accumulation of nanotubes in live tissues relies on side chain variation. A screening of PABA nanomaterials with all possible linker side chains would be the logical extension for generating an ideal oral ingested cargo for mass and efficient delivery.

It determines the parameters of chemical and biological modifications. However, the impact on specific cellular physiology and efficient uptake of nanostructures by carrier molecules based on their chemical modifications cannot be ignored. (2) These organic nanotubes overcome cell physiological, pharmacokinetic barriers and show an efficient cellular uptake in animal cultured cells ^{5,8}. (3) It has no adverse effects in physiology, behaviour and growth of Drosophila ²⁴. These findings further propose that Drosophila might be a potential model for nanobiotechnology. (3) Finally no short-term toxicity, impaired growth of Drosophila larvae and adults after feeding solely on nanotubes containing media was found. During self assembly, chemical modifications of PABA, and their longer stability in the internal organs provides the nanotubes as better-suited and sustainable cargo in live organisms.

### Methods

### FACS and MTT Assay

The cell proliferation was determined by colorimetric assay using 3-(4,5-dimethylthiazol-2yl)-2,5 diphenyltetrazolium bromide (MTT). The assay is based on reductive capacity to metabolize the tetrazolium salt to blue colored formazone. The cultured cells seeded on 96 well microplates nearly 6000 cells/well were incubated for 48 hrs with various concentrations of nanotubes containing fresh media. The medium was changed once with fresh culture medium in 24 hr interval. MTT assay was performed after 1, 2 or 3 days as described earlier (3). Briefly, cells were incubated with 0.5 mg/ml of MTT (Sigma) for 4 hr in a CO2 incubator at 37° C. After the removal of the solution, the purple precipitates were dissolved in DMSO for 20 min at room temperature and the resultant solution was transferred to new 96-well plates. The absorbance was measured at 570 nm using a Lab systems Multiscan RC enzyme-linked immunosorbent assay (ELISA) reader. Each experiment was performed in triplicate, and the means were determined for each time point.

For the FACS assay, cells were pelleted and fixed with ice cold 80% methanol overnight. They were stained with 20 ug/ml of Propidium Iodide (PI) and analysis was done on the MoFlow-Dako Cytomation (Dako, Denmark).

### Cell Culture—

Human HEK-293 and HeLa cells were grown in Dulbecco's modified Eagle's medium (Sigma Chemical, USA) supplemented with 10% fetal bovine serum and common antibiotics (penicillin, kanamycin, and streptomycin) at 1× concentration. Cells were routinely maintained in a humidified atmosphere of 5% CO₂ at 37° C. and were sub-cultured every three days. The cells were seeded at 1×10⁶ per ml, a day prior to treatment in 6 well plates as well as in cover slips for, flow cytometry and confocal microscopy. A day after seeding media was removed completely, adherent cells were given a gentle PBS wash and fresh media was added. The cultures were treated with Dimethyl Sulfoxide (0.1% DMSO) solvent as controls as well as with the DMSO containing organic nanotubes at concentrations of 20 ug/ml to 80 ug/ml. The cultures were harvested after 24 or 48 hrs.

### Confocal Microscopy

For the cytological studies, coverslips were washed with PBS and fixed with 4% para-formaldehyde for 20 mins followed by a PBS wash. They were then mounted on microscopic slides with 80% glycerol. The fluorescence of the nanotubes PNT-A and PNT-B was excited at the 488 nm and 543 nm laser respectively. Confocal images were acquired on Olympus FV1000 laser microscope.

### Estimation of Fluorescence Dyes and Nanotube Accumulation

Nanotubes accumulation was estimated from individual cells and the whole organism as well as different organs by quantifying the amount of fluorescence after excitation at 488 and 543 nm. The amount of nanotube/specified area was counted by determining the gray scale level using Metamorph version 4.6 soft ware. Gray scale, which is defined as brightness of pixel in a digital image is an eight-bit digital signal with 256 possible values ranging from 255 (white) to 0 (black). The mean grey scale values equal the total gray scale values per number of pixels. For estimating the mean gray scale value, we calculated the total (sum of) gray scale values of entire designated area divided by the total pixel of the same area.

### REFERENCES

1. Nam J M, Thaxton C S & Mirkin C A. Nanoparticle-based bio-bar codes for the ultrasensitive detection of proteins. Science 301, 1884-1886 (2003).
2. Tsapis N, Bennett D, Jackson B, Weitz D A & Edwards D A. Trojan particles: large porous carriers of nanoparticles for drug delivery. Proc. Natl. Acad. Sci. USA 99, 12001-12005 (2002).
3. Gao X & Nie S. Molecular profiling of single cells and tissue specimens with quantum dots. Trends Biotechnol. 21, 371-373 (2003).
4. Santra S, Yang H, Holloway P H, Stanley J T & Mericle R A. Synthesis of water-dispersible fluorescent, radio-opaque, and paramagnetic CdS:Mn/ZnS quantum dots: a multifunctional probe for bioimaging. J Am Chem. Soc. 127, 1656-1657 (2005).
5. Liu, Z., Winters, M., Holodniy, M. & Dai, H. J. siRNA delivery into human T cells and primary cells with Carbon Nanotube Transporters. Angew. Chem. Int. Ed. 46, 2023-2027 (2007).
6. Thomas M, Klibanov A M. Conjugation to gold nanoparticles enhances polyethylenimine's transfer of plasmid Dna into mammalian cells. Proc. Natl. Acad. Sci. USA 100, 9138-9143 (2003).
7. Kircher M F, Weissleder R & Josephson, L. A dual fluorochrome probe for imaging proteases. Bioconjug. Chem. 15, 242-248 (2004).
8. Zheng G, Chen J, Li H. & Glickson J D. Rerouting lipoprotein nanoparticles to selected alternate receptors for the targeted delivery of cancer diagnostic and therapeutic agents. Proc. Natl. Acad. Sci. USA 102, 17757-17762 (2005).
9. Peer, D., Park, E. J., Morishita, Y., Carman, C. V. & Shimaoka M. Systemic Leukocyte-Directed siRNA delivery revealing Cyclin D1 as an anti-inflammatory target. Science 319, 627-630 (2008).
10. Modo M. Mellodew K, Cash D, Fraser S E, Meade T J, Price J, Williams S C. Mapping transplanted stem cell migration after a stroke: a serial, in vivo magnetic resonance imaging study. Neuroimage 21, 311-317 (2004).
11. Kang H W, Josephson L, Petrovsky A, Weissleder R. & Bogdanow A Jr. Magnetic resonance imaging of inducible E-selectin expression in human endothelial cell culture. Bioconjug. Chem. 13, 122-127 (2002).
12. Gaolin Liang, Keming Xu, Lihua Li, Ling Wang, Yi Kuang, Zhimou Yang and Bing Xu. Using Congo red to report intracellular hydrogelation resulted from self-assembly of small molecules. Chem. Commun. 40, 4096-4098 (2007).
13. Weissleder, R., Kelly, K., Sun, E. Y., Shtatland, T. & Josephson, L. Cell-specific targeting of nanoparticles by multivalent attachment of small molecules. Nature Biotechnol. 23, 1418-1423 (2005).
14. Moffett R B, Robert A & Skaletzky L L. Antiulcer agents. p-Aminobenzamido aromatic compounds. J. Med. Chem. 14, 963-968 (1971).
15. Schmelke, F. C. & Rubin, M. J. Am. Chem. Soc. 66, 1631 (1944).
16. Plumb J A, Milroy R & Kaye S B. Effects of the pH dependence of 3-(4,5-dimethylthiazol-2-yl)-2,5-diphenyl-tetrazolium bromide-formazan absorption on chemo sensitivity determined by a novel tetrazolium-based assay. Cancer Res. 49, 4435-4440 (1989).
17. Gao L, Nie L, Wang T, Qin Y, Guo Z, Yang D, Yan X. Carbon Nanotube delivery of the GFP gene into mammalian cells. Chem. Bio. Chem. 7, 239-242 (2006).
18. Yadav, J. S. Manoj Kumar Gupta, I. Prathap, Manika Pal Bhadra, Parsi K. Mohan and Bulusu Jagannadh. Synthesis and cellular uptake of cell delivering 2,6-pyridinediylbisalkanamide submicron-sized sheets in HeLa cells. Chem. Commun. 37, 3832-3834 (2007).
19. Kluczyk A. et al., Drug Evolution: p-Aminobenzoic Acid as a Building B. Cur. Med. Chemistry 21, 1871-1892 (2002).
20. Plumb J A., Milroy R. & Kaye, S B. Effects of the pH dependence of 3-(4,5-dimethylthiazol-2-yl)-2,5-diphenyl-tetrazolium bromide-formazan absorption on chemosensitivity determined by a novel tetrazolium-based assay. Cancer Res. 49, 4435-4440 (1989).
21. Pal-Bhadra M., Bhadra U., Kundu, J. & Birchler, J. A. Genetics 169, 2061-2074 (2005).
22. Pal-Bhadra M., Bhadra U. & Birchler J. A. Misregulation of sex-lethal and disruption of male-specific lethal complex localization in Drosophila species hybrids. Genetics 174, 1151-1159 (2006).
23. Reiter L T, Potocki L, Chien S., Gribskov M & Bier E. A Systematic Analysis of Human Disease-Associated Gene Sequences In Drosophila melanogaster, Genome Research 11, 1114-1125 (2001).

1. A nano-tube or nano-rod comprising a drug moiety or its derivative as a structural moiety that further has side chain/s capable of promoting self aggregation, the side chains may be mono or a multiple of alkyl chain/s having even number or odd number of carbon atoms that may be substituted or unsubstituted as simple or complex assemblies.

2. A nano-tube or nano-rod of claim 1 comprising where the said drug moiety or its derivative is p-amino benzoic acid (PABA) derivative, a 4-alkylamido-N-pyridin-2-yl-benzamide of structure 1 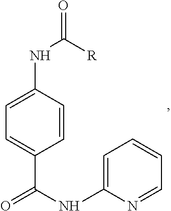
wherein the side chain R may be an alkyl group having carbon atoms 12 to 18 (C=12 to 18) or side chain/s capable of promoting self aggregation, the side chains may be mono or a multiple of alkyl chains having even number or odd number of carbon atoms that may be substituted or unsubstituted for creating simple or complex assemblies.

3. A 4-alkylamido-N-pyridin-2-yl-benzamide of claim 2 comprising a lauric (C=12) or stearic (C=18) side chain.

4. A nanostructure of claim 1 comprising nanotubes or nanorods wherein at least one structural moiety of the nanotube comprises a PABA derivative or a 4-alkylamido-N-pyridin-2-yl-benzamide of structure 1 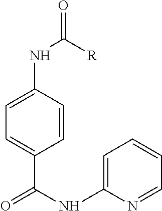
Wherein the side chain R may be an alkyl group having carbon atoms 12 to 18 (C=12 to 18) or side chain/s capable of promoting self aggregation, the side chains may be mono or a multiple of alkyl chains having even number or odd number of carbon atoms that may be substituted or unsubstituted for creating simple or complex assemblies.

5. A 4-alkylamido-N-pyridin-2-yl-benzamide of claim 4 further comprising a PABA derivative with lauric (C=12) or stearic (C=18) side chain.

6. A compound of claim 5 wherein the said PABA derivative comprises a derivative of 4-Amino-N-pyridine-2 yl-benzamide of structure 4

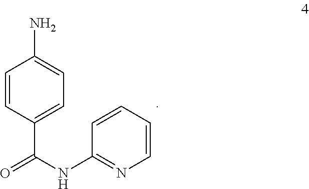 [SMILES: Nc1ccc(C(=O)Nc2ccccn2)cc1]

7. A nanotube or nanorod of claim 4 comprising PABA as a linker for lauric (C=12) and/or stearic (C=18) side chains.

8. A nanotube or nanorod of claim 7 further comprising conjugation of an active ingredient with the said nanotube or nanorod, wherein the active ingredient has a function to perform when absorbed or introduced in the body of a cell or an organism.

9. A nanotube or nanorod of claim 7 when the said function includes a therapeutic action, in vivo tracking, or delivery of genetic material inside a cell or to an organism.

10. A nanotube or nanorod of claim 8 comprising Rhodamine B as an active ingredient.

11. A nanotube or nanorod of claim 9 for the purpose of in vivo tracking.

12. A process of producing nanotubes or nanorods comprising structural moiety of a drug.

13. A process of claim 12 wherein the said structural moiety comprises p-amino benzoic acid (PABA) moiety.

14. A process of claim 13 wherein the said structural moiety of a drug has side chain/s that comprise/s a lauric (C=12) and/or stearic (C=18) side chain.

15. A process of claim 12 comprising adding 4-Dodecanamido-N-(pyridine-2-yl)benzamide and N-(pyridin-2-yl)-4-stearamidobenzamide in a solvent and allowing the nanotubes or nanorods to get self assembled.

16. A process of claim 15 wherein the said solvent is methanol.

17. A process of claim 16 wherein
a. equal weight of 4-Dodecanamido-N-(pyridine-2-yl)benzamide and N-(pyridin-2-yl)-4-stearamidobenzamide are heated with methanol about two times the weight of each of them,
b. upon cooling to room temperature, deionized water in volume equal to the volume of methanol was mixed to obtain the final aggregates.

18. A process of claim 14 when the said structural moiety of a drug has side chain/s that comprise/s a lauric (C=12) and/or stearic (C=18) side chain comprises a 4-alkylamido-N-pyridin-2-yl-benzamide process of preparation of which comprising the steps of:
a. taking a mixture of 4-Amino-N-pyridine-2 yl-benzamide and pyridine in dry THF,
b. adding an acid chloride,
c. refluxing the mixture for overnight,
d. removing pyridine and THF under reduced pressure,
e. washing the residue with saturated NH₄Cl,
f. extracting with dichloro methane (DCM),
g. drying the organic layer over Na₂SO₄ and concentrating under reduced pressure.

19. A process of claim 18 wherein the said 4-alkylamido-N-pyridin-2-yl-benzamide is:
a. 4-Dodecanamido-N-(pyridine-2-yl)benzamide and the said acid chloride used is Dodecanoyl chloride, or/and
b. N-(pyridin-2-yl)-4-stearamidobenzamide is produced when the said acid chloride used is Octadecanoyl chloride.

20. A process of in vivo tracking by using a nanotube or nanorod of claim 4 with intrinsic fluorescence conjugated with a fluorescent moiety.

21. A process of claim 20 wherein the said fluorescent moiety is Rhodamine B.

22. Orally ingestible nanotubes or nanorods of claim 4.

23. Orally ingestible nanotubes or nanorods of claim 22 for intracellular uptake and intercellular delivery of drugs or fluorescent molecule or tracking molecule or an active ingredient/s in cells of insects and human being.

24. Nanotubes or nanorods of claim 23 that have at least one of the following properties:
a. green intrinsic fluorescence under confocal microscope,
b. red fluorescence when Rhodamine B is conjugated or any other fluorescence derived from the molecule conjugated,
c. randomly oriented structures of variable sizes that grow individually during the self-assembly process as seen under 3D Scanning Electron Microscopy images of nanostructures with as well as without Rhodamine or an active molecule,
d. nanotubes with variable sizes under Dynamic light scattering (DLS), the distribution starting with nano-size particles in fresh preparation to about micron size on prolonged storage upto several days, higher magnification images showing the hollow structures,
e. Rhodamine incorporated nanotubes having average heights of the majority of the nanotubes are 3-5 nm as seen in. 3D reconstituted AFM images,
f. soluble in DMSO and ethyl alcohol soluble, but insoluble in water,
g. can be stored in DMSO and an ethyl alcohol/water mixture for long periods without losing their properties during aggregation,
h. biocompatible with at least any one of the following: human embryonic kidney cells, neoplastic HeLa cells, Drosophila larvae, Drosophila adults, Drosophila eggs, Drosophila brain, Drosophila lumen or any other insect cells or human cells,
i. no adverse effect on mortality rates, physiology or locomotion of Drosophila on oral feeding or on external exposure to dry composition,
j. 200 nm in width and 500 nm in length to micron-sized structures in few aggregates,
k. efficient molecular transporters for different biologically important cells with no cytotoxicity,
l. side chain variations leading to changes in nanotube distribution in the body parts, shorter length of the lauric side chain exhibiting greater accumulation of nanostructures; the cells of different discs and larval brains being devoid of any nanotubes containing longer side chains (C=18), a considerable amount of nanotubes containing short chains (C=12) penetrating into the same tissues,
m. overcoming pharmacokinetic barriers and showing an efficient cellular uptake,
n. retention of the original properties of PABA in self-assembly nanotubes increasing PABA concentration in the intestine during oral ingestion, helping in delivery a multifunctional effect including convert by intestinal bacteria it into folic acids, and protecting against strokes, cardiovascular diseases and certain cancers, playing a dual role, directly as a delivery vehicle of therapeutic agents and indirectly by preventing different diseases as a therapeutic agent itself,
o. useful to deliver small regulatory RNA and DNA as therapeutic materials,
p. useful to optimize pill-like properties for orally ingested materials as micropills to deliver biomaterials for effective gene therapy and novel cargoes for molecular populations.

25. (canceled)

26. (canceled)

27. (canceled)

28. 4-alkylamido-N-pyridin-2-yl-benzamide of structure 1 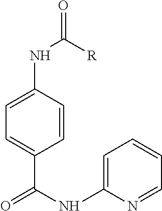
wherein the side chainR may be an alkyl group having carbon atoms 12 to 18 (C=12 to 18) or side chain/s capable of promoting self aggregation, the side chains may be mono or a multiple of alkyl chains having even number or odd number of carbon atoms that may be substituted or unsubstituted for creating simple or complex assemblies.

29. (canceled)

30. N-(Pyridine-2-yl)-4-dodecanamido-benzamide of structure (1a):

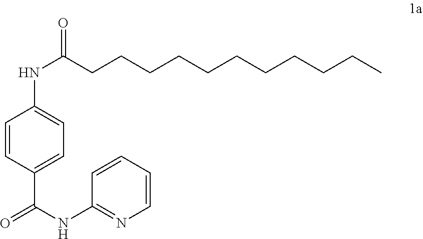 [SMILES: CCCCCCCCCCCC(=O)Nc1ccc(C(=O)Nc2ccccn2)cc1]

31. N-(Pyridin-2-yl)-4-stearamidobenzamide of structure (1b):

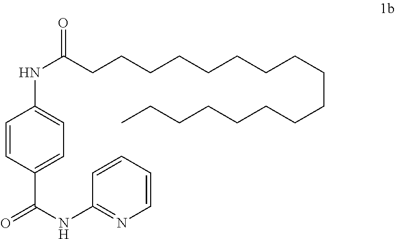 [SMILES: CCCCCCCCCCCCCCCCCC(=O)Nc1ccc(C(=O)Nc2ccccn2)cc1]

32. A nano-rod or nanotube of claim 1, that are capable of being taken up by an organism after administration either for application for in vivo tracking or/and conjugating with one or more of an active for administration to an organism for carrying out a certain function
